(19) United States
(12) Patent Application Publication
(10) Pub. No.: US 2020/0368420 A1
(43) Pub. Date: Nov. 26, 2020

(54) SYSTEMS AND METHODS FOR EXCHANGING SMALL MOLECULES WITH FLUID

(71) Applicant: United Therapeutics Corporation, Silver Spring, MD (US)

(72) Inventor: Anna M. Galea, Stow, MA (US)

(73) Assignee: United Therapeutics Corporation, Silver Spring, MD (US)

(21) Appl. No.: 16/882,022

(22) Filed: May 22, 2020

Related U.S. Application Data
(60) Provisional application No. 62/852,517, filed on May 24, 2019.

(51) Int. Cl. A61M 1/16

(52) U.S. Cl. CPC A61M 1/1678; A61M 1/1698; A61M 2205/7527; A61M 2202/0225; A61M 2202/0208

(57) ABSTRACT

An apparatus for exchanging small molecules with a fluid includes a small-molecule conduit for providing a first fluid having a first type of small molecule, a target fluid conduit for providing a target fluid having a second type of small molecule therein, and a carrier fluid conduit for providing a carrier fluid that is configured (i) to receive at least some of the first type of small molecule from the first fluid and transfer at least some of the first type of small molecule to the target fluid and (ii) to receive at least some of the second type of small molecule from the target fluid and transfer at least some of the second type of small molecule to the first fluid. The apparatus further includes an exchange module having an exchange chamber in fluid communication with the small-molecule conduit, the target fluid conduit and the carrier fluid conduit.

### CROSS-REFERENCE TO RELATED APPLICATIONS

This application claims priority to U.S. Provisional Application No. 62/852,517, filed May 24, 2019, which is hereby incorporated by reference in its entirety.

### BACKGROUND

The present invention relates generally to systems and methods for small molecule exchange with a fluid, preferably for gas exchange with a physiological fluid, and more preferably for oxygenating and removing carbon dioxide from a physiologic fluid.

The main function of lungs is to transfer oxygen from the atmosphere into the blood and expel carbon dioxide therefrom to the atmosphere. For patients with diseased or damaged lungs, this exchange of gas is compromised and there are few treatment options. Some of the most common diseases leading to end-stage lung failure include, inter alia, chronic obstructive pulmonary disease (COPD), cystic fibrosis (CF), idiopathic pulmonary fibrosis (IPF), and pulmonary hypertension (PH). There are also many people suffering from lung cancer and poor lung function due to years of smoking who are not eligible for a lung resection or lung transplantation.

Lung transplantation remains the main therapy for chronic irreversible respiratory failure. However, lung transplantation is not very common; only about 2,000 procedures are performed each year in the United States. The most common indications for lung transplantation include COPD, CF, IPF, and PH. Patients with lung cancer are not candidates for transplantation because the use of immunosuppression could potentially cause the cancer to spread. Lung transplant candidates can die waiting for an organ donor since the average waiting time period may exceed two years. The overall results are not ideal due to the extensive surgery required, deterioration of the patient's condition during the waiting period, the complications of chronic immunosuppression, infection, and the development of chronic rejection. Also, many patients with chronic lung disease tend to be older individuals who are poor candidates for transplantation because they do not tolerate immunosuppression.

Another option for patients suffering from diseased or damaged lungs may be to utilize an enriched oxygen supply, frequently in conjunction with a ventilator. However, this has been shown to create dependency and a host of other ventilator-related disorders.

The concept of using an artificial lung in clinical medicine to take over the gas exchange function of diseased or damaged lung(s) dates back to the development of the heart-lung machine in 1954. Cardiopulmonary bypass (CPB) is a technique used to take over the function of the heart and lungs during surgery by regulating the circulation of blood and oxygen within a person's body. The artificial lung may provide short-term pulmonary support during extensive operations on the heart.

Over the last few decades, several conventional mechanical-assisting devices have been developed to treat diseased or damaged lung(s) acute reversible respiratory failure due to acute respiratory distress syndrome (ARDS). Conventional systems have also been developed for short-term pulmonary support (e.g., days to a few weeks). These systems include extracorporeal membrane oxygenation (ECMO) devices, extracorporeal carbon dioxide removal (ECCO₂R) devices, and intravascular oxygenators (IVOX) devices.

Although conventional ECMO and IVOX systems have been used for aiding patients with diseased or damaged lung(s), they are both one-stage systems with distinct drawbacks. ECMO devices produce significant complication rates and typically do not provide a significant improvement. IVOX devices may alleviate some of the problems associated with ECMO devices. However, the gas exchange area of IVOX devices may be too small and the device may not provide the needed total support for gas exchange. Also, IVOX devices may not take away excess carbon dioxide leftover within the system. Finally, conventional one-stage IVOX systems typically include membranous or fibrous components used for oxygenation. Typically, a bundle of hollow fibers may be used as the oxygenating element. Exposing blood to the large artificial surface area needed for gas exchange often causes blood activation and thrombogenesis. ECCO₂R and ECMO are also one-stage systems and may be limited by the inclusion of fibers that come in contact with blood thereby causing blood activation and thrombogenesis.

Generally, these devices use a membrane that can selectively allow the transport of gas molecules. Unfortunately, the interaction of the blood with the membrane results in the blood laying down a protein layer on the membrane in the start of the blood's coagulation cascade. The protein layer renders the membrane less efficient to the gas transport. As coagulation progresses further, the device becomes less effective and greater pressures are required to pump the blood through the device, eventually reaching the point where blood cells lyse as they are pumped to and through the device, creating further problems.

There are advances that can be applied to materials that would decrease or even eliminate these issues. However, the membrane material poses the key problem in these devices because it has proven difficult to apply these advances to a gas permeable surface while maintaining efficiency of gas transfer.

There has also been some research in utilizing an oxygen-carrying liquid to bring oxygen directly to the blood, however this research has focused on using small bubbles of liquid that are injected into the blood and then removed using a selective filter. When bubbles of fluid are injected into the blood, the system typically requires a means for pulling the bubbles out of the blood before it flows back into a user, which may also cause blood activation. These systems typically do not appreciably decrease the amount of carbon dioxide in the blood.

### SUMMARY OF THE INVENTION

One aspect of the disclosure relates to an apparatus for exchanging small molecules with a fluid. The apparatus includes a small-molecule conduit for providing a first fluid having a first type of small molecule, a target fluid conduit for providing a target fluid having a second type of small molecule therein, and a carrier fluid conduit for providing a carrier fluid that is configured to at least one of: (i) receive at least some of the first type of small molecule from the first fluid and transfer at least some of the first type of small molecule to the target fluid and (ii) receive at least some of the second type of small molecule from the target fluid and transfer at least some of the second type of small molecule to the first fluid. The apparatus further includes an exchange module having an exchange chamber in fluid communication with the small-molecule conduit, the target fluid conduit and the carrier fluid conduit to receive the first fluid, the carrier fluid, and the target fluid with the exchange chamber, wherein the exchange chamber is configured (i) to position the first fluid relative to the carrier fluid to permit the transfer of at least one of the first type of small molecule and the second type of small molecule between the first fluid and the carrier fluid and (ii) to position the carrier fluid relative to the target fluid to permit the transfer of at least one of the first type of small molecule and the second type of small molecule between the target fluid and the carrier fluid.

Another aspect of the disclosure relates to a method of exchanging small molecules with a fluid. The method includes flowing, through an exchange chamber of an exchange module on a first side of a membrane, a first fluid comprising a first type of small molecules, flowing, through the exchange chamber of the exchange module on a second side of the membrane, a target fluid having a second type of small molecules therein, and flowing, through the exchange chamber of the exchange module on the second side of the membrane and between the target fluid and the membrane, a carrier fluid that at least one of: (i) receives through the membrane at least some of the first type of small molecules from the first fluid and transfers at least some of the first type of small molecules to the target fluid and (ii) receives at least some of the second type of small molecules from the target fluid and transfers through the membrane at least some of the second type of small molecules to the first fluid. The first fluid, the target fluid, and the carrier fluid are flowed simultaneously through the exchange chamber of the exchange module.

Another aspect of the disclosure relates to a method of exchanging small molecules with a fluid. The method includes providing a primary exchange module configured to: receive a first fluid having a first type of small molecule therein; receive a carrier fluid having second type of small molecule therein, and transfer at least one of: (i) the first type of small molecule from the first fluid to the carrier fluid and (ii) the second type of small molecule from the carrier fluid to the first fluid to create at least one of a carrier fluid loaded with the first type of small molecule and a first fluid loaded with the second type of small molecule. The method further includes providing a secondary exchange module configured to: receive the carrier fluid loaded with the first type of small molecule; receive a target fluid having the second type of small molecule therein; and transfer at least one of: (i) the first type of small molecule from the carrier fluid loaded with the first type of small molecule to the target fluid and (ii) the second type of small molecule from the target fluid to the carrier fluid to create at least one of a target fluid loaded with the first type of small molecule and a carrier fluid loaded with the second type of small molecule. The method further includes implanting the secondary exchange module within a body of a patient, and positioning the primary exchange module external to the body of the patient.

### BRIEF DESCRIPTION OF THE DRAWINGS

FIG. 1 is a block diagram showing the primary components of one embodiment of a system, having a primary and secondary exchange module, for exchanging small molecules with a fluid;

FIG. 2 is a three-dimensional view showing in further detail the primary exchange module shown in FIG. 1;

FIG. 3A is a schematic end-view showing one example of the structure of the microfluidic channel of the secondary exchange module shown in FIG. 1;

FIG. 3B is a schematic top-view of the microfluidic channel shown in FIG. 3A showing one example of the flow of two fluids therein;

FIG. 3C is a schematic bottom-view of the microfluidic channel shown in FIG. 3A showing one example of the flow of two fluids therein;

FIG. 4 is a three-dimensional front-view showing in further detail one embodiment of the microfluidic channels shown in FIG. 3A-C;

FIG. 5 is a schematic front-view showing one example of the two-stage system for exchanging small molecules with a fluid according to the embodiment of FIG. 1;

FIG. 6 is a front view of a second embodiment of a secondary exchange module.

FIGS. 7A-7D show cross-sectional views of an embodiment of the fluid channel shown in FIG. 6.

FIG. 8 is a diagram depicting various examples of the positioning of the exchange modules of the system of FIG. 1 relative to a body of a patient;

FIG. 9 is a block diagram showing the primary components of another embodiment of a system for exchanging small molecules with a fluid, having a single exchange module;

FIG. 10 is a schematic diagram showing one example of the two-stage system for exchanging small molecules with a fluid according to the embodiment of FIG. 9;

FIG. 11 is a cross-sectional view of an exchange module of the system according to the embodiment of FIG. 9;

FIG. 12A-C are cross-sectional views of various embodiments of an exchange module of the system according to the embodiment of FIG. 9, having varying dimensions;

FIG. 13A-13D are cross-sectional views of various embodiments of an exchange module of the system according to the embodiment of FIG. 9.

### DETAILED DESCRIPTION

Described herein are various systems and methods for exchanging small molecules with a fluid. In one embodiment, a two-stage system utilizes first and second exchange modules. In another embodiment, a single-stage system is provided by a single exchange module.

For ease of reference, embodiments in the present disclosure are described specifically with respect to oxygenating and removing carbon dioxide from a physiological fluid. However, it is to be understood that the systems and methods described herein apply to and can be used for other applications for exchanging small molecules with a fluid. Other potential applications to this system include hemodialysis, ultrafiltration, plasmapheresis, and the removal of bacteria from the blood stream. While the present application describes a gas having oxygen as a first fluid, a physiological fluid (such as blood) as a target fluid, and a carrier fluid that serves as an intermediary fluid, and it is to be understood that the systems and methods can be also used for other fluids. For example, in hemodialysis, the first fluid can be the dialysate, the carrier fluid can be plasma, and the target fluid can be blood. In ultrafiltration, the first fluid can be a liquid or a gas, the carrier fluid can be plasma or another liquid, and the target fluid can be blood. In plasmapheresis, the first fluid can be a liquid specially formulated to remove toxins, the carrier fluid can be plasma, and the target fluid can be blood.

Also for ease of reference, in the embodiments described herein, a two-way transfer of small molecules is described, particularly, delivering oxygen to and removing carbon dioxide from a physiological fluid. It is to be understood, however, that systems described herein can also be configured to accommodate one-way transfer alone, such as providing oxygen to the physiological fluid or removing carbon dioxide from the physiological fluid. In such embodiments, the small molecules of the first fluid are transferred to the carrier fluid and then to the target fluid, or the small molecules are transferred from the target fluid to the carrier fluid and then to the first fluid. As a further example, in hemodialysis, the target fluid (blood) may transfer waste products to the dialysate. Other such configurations are envisioned for one-way or two-way transfer in various applications.

### Two-Stage Exchange System

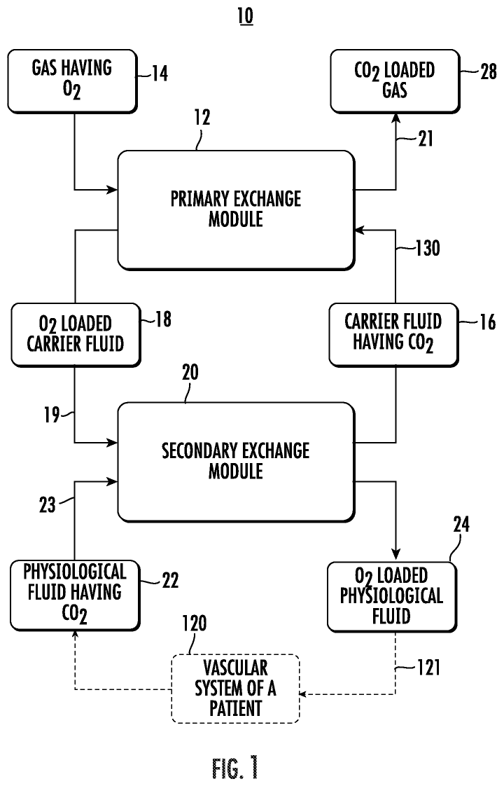

Referring to FIG. 1, one embodiment of two-stage system 10 for exchanging small molecules with a fluid, and particularly for oxygenating and removing carbon dioxide from a physiological fluid, is shown. The features and characteristics of system 10 may be similar to those of the two-stage system described in U.S. Pat. No. 8,574,309 entitled “Two-Stage System and Method for Oxygenating and Removing Carbon Dioxide from a Physiological Fluid,” which is hereby incorporated by reference herein in its entirety.

As shown in FIG. 1, system 10 includes primary exchange module 12 configured to receive a first fluid and a carrier fluid, for example, gas 14 having oxygen therein and carrier fluid 16 having carbon dioxide therein. In one example the gas having oxygen therein may include ambient air, an oxygen gas, or any gas having oxygen therein. Carrier fluid 16 is preferably immiscible with respect to physiological fluid 22 and may be made of a perfluorocarbon, such as a perfluorodecalin (C₁₀F₁₈), or similar type compound known to those skilled in the art, that prevents carrier fluid 16 from mixing with physiological fluid 22 having carbon dioxide therein (discussed below). Primary exchange module 12 transfers oxygen from gas 14 having oxygen therein to carrier fluid 16 and transfers the carbon dioxide in carrier fluid 16 to gas 14 to create oxygen loaded carrier fluid 18 and carbon dioxide loaded gas 28. Carbon dioxide loaded gas is preferably expelled from primary exchange module 12, as shown at 21.

System 10 also includes secondary exchange module 20 which receives the carrier fluid and a target fluid (i.e., a fluid that is the target for the small molecule exchange), for example, oxygen loaded carrier fluid 18 from primary exchange module 12, indicated at 19, and physiological fluid 22 having carbon dioxide therein indicated at 23. Physiological fluid 22 may include blood, serum, or any similar type physiological fluid having carbon dioxide therein. In one example, physiological fluid 22 having carbon dioxide therein may be received from vascular system of a patient 120. Secondary exchange module transfers the oxygen from oxygen loaded carrier fluid 18 to physiological fluid 22 and transfers carbon dioxide from physiological fluid 22 to produce oxygen loaded physiological fluid 24 and carrier fluid 16 having carbon dioxide therein. Oxygen loaded physiological fluid 24, which now has carbon dioxide removed, may then be transferred to vascular system of a patient 120, as shown at 121. Carrier fluid 16, having carbon dioxide therein, is transferred to primary exchange module 12, as shown at 130.

The result is system 10 receives physiological fluid 22 having carbon dioxide therein, effectively removes carbon dioxide therefrom and loads physiological fluid 22 with oxygen. Oxygen loaded physiological fluid 24 may be then transferred to vascular system of patient 120. Thus, system 10 can be used to effectively assist or replace the function of diseased or damaged lung(s) discussed in the Background section above. In one embodiment, system 10 may be used as an artificial lung.

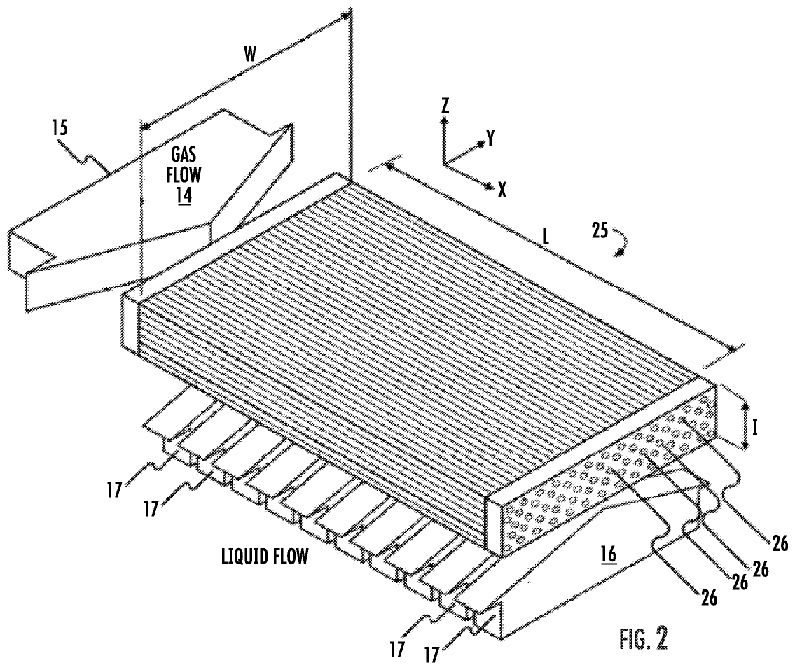

Primary exchange module 12 preferably includes at least one array, e.g., array 25, shown in FIG. 2, which includes a plurality of hollow fibers 26 which receive gas 14 having oxygen therein. In one example, gas 14 enters hollow fibers 26 in the direction indicated by arrow 15 and flows through hollow fibers 26. Array 25 is preferably in fluidic communication with carrier fluid 16 having carbon dioxide therein. In one design, carrier fluid 16 having carbon dioxide flows into array 25, in the direction indicated by arrows 17 and travels about and in close proximity to each of the hollow fibers 26, e.g., in and around the hollow fibers, as the carrier fluid 16 flows substantially perpendicular to the flow of the gas 14. Hollow fibers 26 efficiently transfer the oxygen in gas 14 to the carrier fluid 16 and efficiently transfer the carbon dioxide in carrier fluid 16 to the gas inside hollow fibers 26.

Primary exchange module 12 may include a plurality of arrays 25. Each of arrays similarly includes hollow fibers 26 as discussed above. In this example, carrier fluid 16 having carbon dioxide therein enters primary exchange module 12 and then flows in between the plurality of arrays. Carrier fluid 16 then travels in an upward and downward direction into the arrays and flows in between and about hollow fibers 26 of the arrays. When carrier fluid 16 travels in between, about, and in close proximity to, hollow fibers 26, hollow fibers 26 efficiently transfer the oxygen in gas 14 to carrier fluid 16 and transfer the carbon dioxide in carrier fluid 16 to the gas inside hollow fibers 26 to create oxygen loaded carrier fluid 18 and carbon dioxide loaded gas 28, as depicted in FIG. 1.

In one embodiment, the distance between one or more, or each of, hollow fibers 26 is preferably configured to provide the efficient transfer of the oxygen from the gas having oxygen therein to the carrier fluid and the transfer of the carbon dioxide from the carrier fluid to the gas as discussed above. In one example, the distance between one or more, or of each hollow fiber 26 is preferably smaller than or equal to the outer diameter hollow fibers 26. In one example, the distance between the hollow fibers 26 is about 100 microns and outer diameter distances of the hollow fibers are about 125 microns.

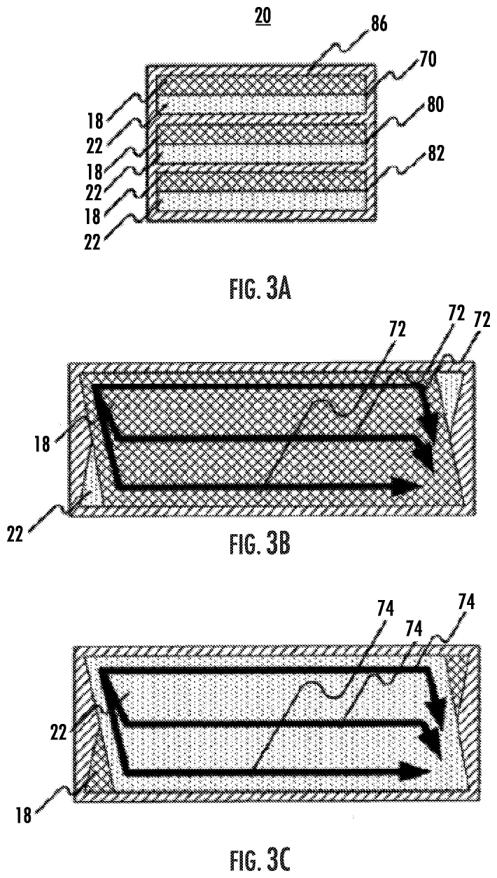

Referring now to secondary exchange module 20, the secondary exchange module 20 preferably includes at least one microfluidic channel 70, as shown in FIG. 3A, which is designed to create a parallel flow of oxygen loaded carrier fluid 18 and physiological fluid 22 having carbon dioxide therein. The parallel flow of oxygen loaded carrier fluid 18 and physiological fluid 22 having carbon dioxide therein provides the efficient transfer of oxygen and carbon dioxide between carrier fluid 18 and physiological fluid 22 to create oxygen loaded physiological fluid 22, and carrier fluid 16 having carbon dioxide therein, as discussed above.

In one example, channel 70 shown in FIG. 3A is preferably designed with a predetermined height to create the parallel flow of oxygen loaded carrier fluid 18 and physiological fluid 22 having carbon dioxide therein. Preferably, the predetermined height is less than or equal to about 1 mm. The height of channel 70 may also be designed to reduce the Reynolds number such that the effective viscosity of oxygen loaded carrier fluid 18 and physiological fluid 22 is increased to maintain the parallel flow. The length of channel 70 is preferably long enough such that the parallel flow of oxygen loaded carrier fluid 18 and physiological fluid 22 having carbon dioxide provides for efficiently transferring the oxygen and carbon dioxide as discussed above. In one example, length of channel 70 is about 0.5 mm to about 2.5 cm, although channel 70 may be any length as known by those skilled in the art.

FIG. 3B shows a top view of one example of flow 72 of oxygen loaded carrier fluid 18 in parallel and flowing over physiological fluid 22 having carbon dioxide therein. FIG. 3C shows an example of a bottom view of channel 70 depicting flow 74 of physiological fluid 22 flowing parallel to and, in this example, under oxygen loaded carrier fluid 18. In other examples physiological fluid 22 may flow in parallel and over oxygen loaded carrier fluid 18.

In one design, secondary exchange module 20 includes a plurality of channels, e.g., channels 70, 80, and 82 of FIG. 3A. In this example, channel 70 creates a parallel flow of oxygen loaded carrier fluid 18 and physiological fluid 22 having carbon dioxide therein, as discussed above. Similarly, channel 80 creates a parallel flow of oxygen loaded carrier fluid 18 and physiological fluid 22 having carbon dioxide therein, and channel 82 creates a parallel flow of oxygen loaded carrier fluid 18 and physiological fluid 22 having carbon dioxide therein. In one example, the plurality of channel 70, 80, and 82 are housed in chamber 86.

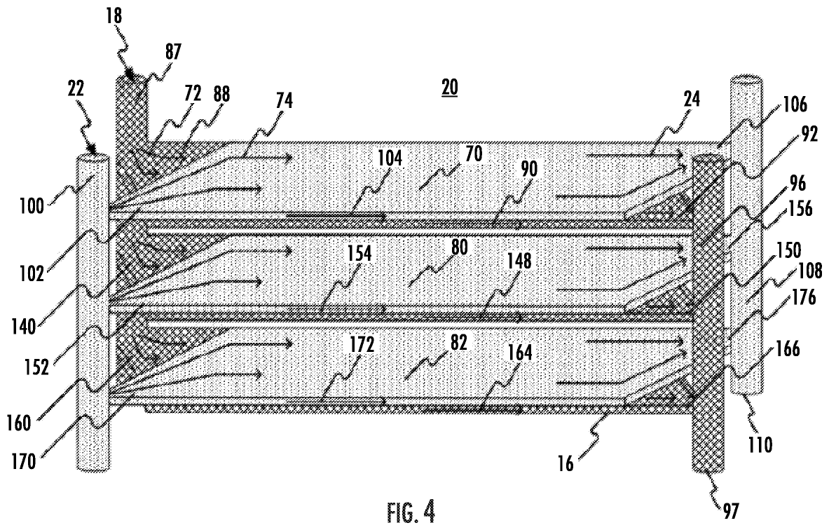

Referring to FIG. 4, where like parts have been given like numbers, one example of the structure of secondary exchange module 20 with channels 70, 80, and 82 is shown. In this example, oxygen loaded carrier fluid 18 flows into microtubule 87, enters channel 70 at inlet plenum 88, and then flows in the direction shown by arrows 90 to the far end of channel 70 where it exits channel 70 as carrier fluid 16 having carbon dioxide therein via outlet plenum 92. Carrier fluid 16 having carbon dioxide therein then flows into microtubule 96, travels down microtubule 96, and then exits secondary exchange module 20 via outlet 97. Carrier fluid 16 having carbon dioxide therein is then transferred to primary exchange module 12 and processed as discussed above. As shown in FIG. 4, physiological fluid 22 having carbon dioxide therein, e.g., from vascular system of a patient 120 flows into microtubule 100, enters channel 70 via inlet plenum 102, travels in the direction indicated by arrows 104 to the far end of channel 70 where it exits channel 70 as oxygen loaded physiological fluid 22 via a outlet plenum 106. Oxygen loaded physiological fluid 24 then flows into microtubule 108, travels down microtubule 108, and then exits secondary exchange module 20 via outlet 110. Oxygen loaded physiological fluid 22 may then be transferred back to vascular system of a patient 120.

Similarly, oxygen loaded carrier fluid 18 may flow into microtubule 87, enter channels 80, 82 at inlet plenums 140, 160, respectively, and then flows in the direction shown by arrows 148, 164 to the far end of channels 80, 82 where it exits channels 80, 82 as carrier fluid 16 having carbon dioxide therein via outlet plenums 150, 166, respectively. Carrier fluid 16 having carbon dioxide therein then flows into microtubule 96, travels down microtubule 96, and exits secondary exchange module 20 via outlet 97. Carrier fluid 16 having carbon dioxide therein may be then transferred to primary exchange module 12 (shown in FIG. 1) where it is processed as discussed above. Physiological fluid 22 having carbon dioxide therein, e.g., from vascular system of a patient 120, flows into microtubule 100, enters channels 80, 82 via inlet plenums 152, 170, respectively, travels in the direction indicated by arrows 154, 172 to the far end of channels 80, 82 where it exits channels 80, 82 as oxygen loaded physiological fluid 22 via outlet plenums 156, 176, respectively. Oxygen loaded physiological fluid 24 then flows into microtubule 108, travels down microtubule 108, and then exits secondary exchange module 20 via outlet 110. Oxygen loaded physiological fluid 22 may then be transferred back to vascular system of a patient 120.

As shown in FIG. 4, microfluidic channels 70, 80, and/or 82 create a parallel flow of oxygen loaded carrier fluid 18 and physiological fluid 22 having carbon dioxide therein. The parallel flow provides an efficient transfer of oxygen from oxygen loaded carrier fluid 18 to physiological fluid 22 having carbon dioxide therein and an efficient transfer of the carbon dioxide from physiological fluid 22 to the carrier fluid 16 to create oxygen loaded physiological fluid 22 and carrier fluid 16 having carbon dioxide therein. As discussed above, oxygen-loaded physiological fluid 22 has the carbon dioxide removed therefrom. Oxygen loaded physiological fluid 22 may then be transferred to vascular system of a patient 120, and carrier fluid 16 having carbon dioxide therein is transferred to primary exchange module 12, as discussed above.

In one embodiment, microfluidic channel 70, shown in FIGS. 3A, and 4, and/or microfluidic channels 80 and 82 may include opposing surfaces which may be coated with, or made of, a material configured to stabilize and further separate the parallel flow of oxygen loaded carrier fluid 18 and physiological fluid 22 having carbon dioxide therein. For example, one of the opposing surfaces may be coated, or made of, a material having hydrophobic properties which attract oxygen loaded carrier fluid 18 and repel physiological fluid 22. The other of surfaces may be coated with, or made of, a material having hydrophylic properties which attract physiological fluid 22 having carbon dioxide therein and repel the oxygen loaded carrier fluid 18. Such a design stabilizes and further separates the parallel flow of oxygen loaded carrier fluid 18 and physiological fluid 22 having carbon dioxide therein. In one example, one surface is coated with, or made of a fluorinated compound, such as polytetrafluoroethylene and another surface is coated with, or made of polyhydroxyethylmethacrylate. The surfaces may be coated with, or made of, or similar type materials known to those skilled in art.

Preferably, carrier fluid 18 and physiological fluid 22 are immiscible with each other to stabilize and further separate the parallel flow thereof. Microfluidic channel 70 with opposing surfaces and/or microfluidic channels 80, 82 (which similarly have opposing surfaces) preferably includes a predetermined shape which increases the surface area thereof in relation to the cross-sectional area of the microfluidic channel to stabilize and further separate the parallel flow of carrier fluid 18 and physiological fluid 22. For example, channel 70 and/or channels 82, 82 may have a scalloped shape, a circular shape, an offset circular shape, or a rectangular shape. Other shapes that increase surface area relative to cross-sectional area will be known to those skilled in the art.

Preferably, microfluidic channel 70 and/or microfluidic channels 80, 82 are made of a bio-compatible material, such as polycarbonate, polyetherimide or similar type materials. In one design, carrier fluid 16 having carbon dioxide therein and/or oxygen loaded carrier fluid 18 may include a perfluorocarbon that prevents carrier fluid 16 from mixing with physiological fluid 22 having carbon dioxide therein.

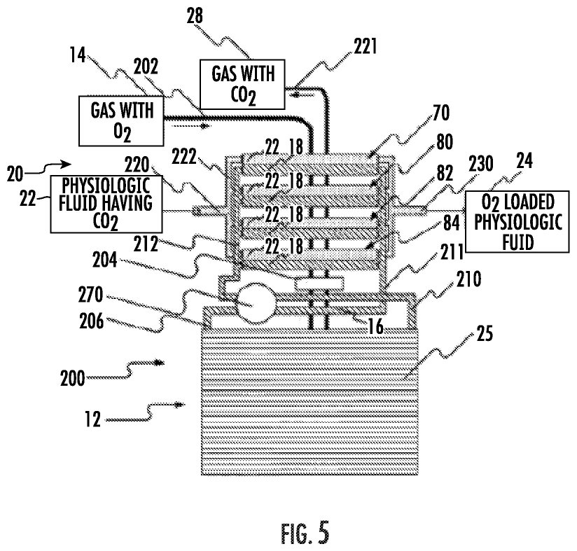

One embodiment of system 10 is shown in FIG. 5. In this example, primary exchange module 12 includes gas and fluidic distribution subsystem 200 which may include gas inlet and line 202 coupled to array 25 having hollow fibers 26 as discussed above with reference to FIG. 2. In one example, subsystem 200 may include blower 204, or similar type device, which draws gas 14 have oxygen therein and delivers it to array 25. In another embodiment, bellows may be used. Subsystem 200 also includes inlet 206 which is in fluidic communication with secondary exchange module 20. Inlet 206 receives a flow of carrier fluid 16 having carbon dioxide therein from secondary exchange module 20. As shown in FIG. 5, gas and fluidic distribution subsystem 200 also preferably includes fluidic outlet 210 which transfers oxygen loaded carrier fluid 18 via microtubule 212 to microfluidic channels 70, 80, and 82 of secondary exchange module 20, similar as discussed above with reference to FIGS. 3A-4. Gas and fluidic distribution subsystem 200 also includes gas outlet 221 which expels carbon dioxide loaded gas 28 to the environment. Secondary exchange module 20 preferably includes fluidic inlet 220 which receives physiological fluid 22 having carbon dioxide therein. Inlet 220 is in fluidic communication via microtubule 222 to each of channels 70, 80, and 82. Microfluidic channels 70, 80, and 82 create a parallel flow of physiological fluid 22 having carbon dioxide therein and oxygen loaded carrier fluid 18, similar as discussed above with reference to FIGS. 3A-4, to provide the efficient transfer of oxygen from the oxygen loaded carrier fluid 18 to physiological fluid 22 and the transfer of carbon dioxide from physiological fluid 22 to the carrier fluid 18 to create oxygen loaded physiological fluid 24. Secondary exchange module 20 preferably includes outlet 230 in fluidic communication with channels 70, 80, and 82. Outlet 250 is preferably coupled to a vascular system of a patient 120 to deliver oxygen loaded physiological fluid 24 to vascular system of the patient 120. Secondary exchange module 20 also includes fluidic outlet 211 in fluidic communication with inlet 206 of primary exchange module 12 which transfers carrier fluid 16 having carbon dioxide therein to primary exchange module 12. In one example, pump 270 may be used to drive the transfer of carrier fluid 16 having carbon dioxide therein to primary exchange module 12.

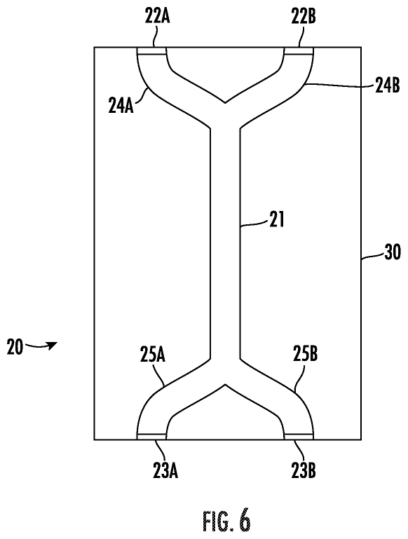

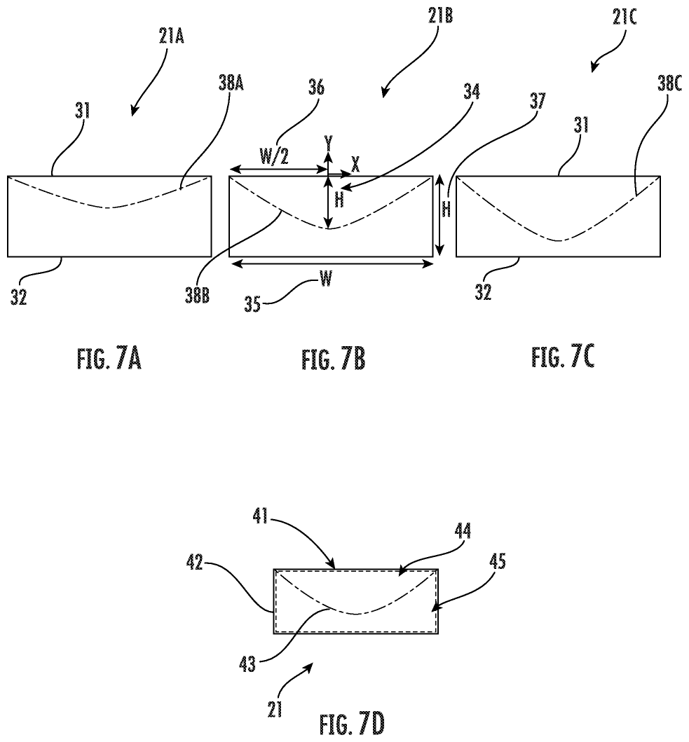

FIGS. 6 and 7A-7D depict an alternative embodiment of a secondary exchange module 20. The features and characteristics of this embodiment of exchange module 20 may be similar to those as described in U.S. Provisional Patent Application No. 62/664,494 entitled “Apparatus and Method for Controlling Fluid Flow,” which is hereby incorporated by reference herein in its entirety.

FIG. 6 shows a front view of a secondary exchange module 20 configured to allow the flow of multiple fluids in the same fluid channel 21. The module 20 is particularly useful for flowing multiple fluids in cases in which one of the fluids is blood. Module 20 includes a housing 30 that can support the fluid channel 21 configured to facilitate two fluids, such as a carrier fluid and a target fluid, flowing through the channel.

The housing 30 also can support structure for supplying fluids to the fluid channel 21. For example, a carrier fluid input 22A and a target fluid input 22B can be in fluid communication with an input channel 24A and an input channel 24B, respectively. In turn, the input channel 24A and the input channel 24B are in fluid communication with the fluid channel 21.

Each of inputs 22A and 22B can be configured, by conventional means, for connection with a respective fluid source (not shown) (e.g., an IV bag, etc.), such that fluid inputs 22A and 22B receive a carrier fluid and a target fluid, respectively, from the fluid sources. As shown in FIG. 2, the carrier and target fluids each flow through respective input channels 24A and 24B into fluid channel 21.

The housing 30 also can support structure for receiving fluids from the fluid channel 21. For example, an output channel 25A and an output channel 25B are in fluid communication with the fluid channel 21. In turn, the output channel 25A and the output channel 25B are in fluid communication with a carrier fluid output 23A and a target fluid output 23B. Output channels 25A and 25B receive respective fluids from the fluid channel 21. The output channels 25A and 25B provide the respective fluids to the carrier fluid output 23A and the target fluid output 23B, respectively. Fluid outputs 23A and 23B are configured to exit a fluid flowing out of the apparatus, for example, a carrier fluid loaded with a type of small molecule transferred from the target fluid and a target fluid loaded with a type of small molecule transferred from the carrier fluid. The fluid outputs 23A and 23B can be configured, by conventional means, for connection with further tubing or other receptacles for the fluids.

Preferably, the housing 30 supports the fluid channel 21 such that fluid can be pumped to flow through the fluid channel such that the system (and specifically the fluid flow) is unaffected by gravity. The fluid channel 21 is configured to allow flow of multiple fluids (e.g., a carrier fluid and a target fluid) while substantially maintaining fluid separation. Thus, molecular transport can be facilitated between the two fluids without fluid intermixture occurring.

The fluid channel 21 can be formed in a variety of configurations. For example, it can be a flexible or rigid channel. Additionally, it can have a variety of cross-sectional shapes, but a rectangular cross-sectional shape with four sides is preferred. The fluid channel 21 can be formed of any suitable material for transporting biomaterials.

FIGS. 7A-7C show a cross-sectional view of an embodiment of the fluid channel 21. For example, as shown in FIG. 7A-7C, fluid channel 21 has a rectangular cross section. Fluid channel 21 has a width (w) 35 and a half-width (w/2) 36, as well as a height (h) 37. Further, fluid channel 21 has at least one first internal surface 31 and at least one second internal surface 32.

In some embodiments, the at least one first internal surface 31 has an affinity to the carrier fluid, and the at least one second internal surface 32 has an affinity to the target fluid. The affinity of the internal surfaces 31, 32 can be established in a variety of ways. For example, the affinity can be established by the material of the corresponding portion of the fluid channel 21. For example, a hydrophilic surface could be made of hydrogels, polyamides, or hydroxylated polyurethanes. As another example, a hydrophobic surface could be made of polytetrafluoroethylene or polymethylene. Alternatively, the affinity can be established by a treatment, such as a coating, applied to the interior of the fluid channel 21. Such treatments include plasma or corona treatments or coating a surface with hydrogels, polyamides, or hydroxylated polyurethanes (to create a hydrophilic surface) or coating a surface with polytetrafluoroethylene or polymethylene (to create a hydrophobic surface. For example, the affinity of each internal surface can be established by applying a first coating on the at least one first internal surface and applying a second coating to the at least one second internal surface. As a more specific example, the above substances can be applied in any suitable order, with appropriate masking (e.g., apply or coat a first coating on the first internal surface, mask the first coating, and apply or coat a second coating on the second internal surface).

As one example of the affinities of the surfaces for the fluids, the at least one first internal surface 31 can be configured to be one of oleophobic and hydrophobic and the at least one second internal surface 32 can be configured to be the other of oleophobic and hydrophobic. In a further example, the at least one first internal surface 31 can be configured to be one of hydrophilic and hydrophobic and the second internal surface can be configured to be the other of hydrophilic and hydrophobic 32. For example, for hydrophilicity a contact angle with water of no more than 50 degrees is preferred, and for hydrophobicity a contact angle with water of more than 110 degrees is preferred.

When multiple immiscible fluids flow in fluid channel 21, a fluid interface 38 is created by a carrier fluid and a target fluid. Depending on parameters, such as the configuration of the fluid channel 21, the flow rates, and the fluids used, the fluid interface may occur at different locations within the fluid channel 21. For example, the parameters may cause the fluid interface 38A to exist at the location in the fluid channel 21A shown in FIG. 7A. As another example, the parameters can cause the fluid interface 38B to exist at the location in the fluid channel 21B shown in FIG. 7B. As a still further example, the parameters can cause the fluid interface 38C to exist at the location in the fluid channel 21C shown in FIG. 7C.

In various embodiments, different internal surface portions of the fluid channel are each configured to have affinities to different fluids. For example, a first internal surface portion has an affinity to a carrier fluid (e.g., an aqueous fluid) and a second internal surface portion has an affinity to a target fluid (e.g., an oleic fluid). In some embodiments, the first internal surface portion has an affinity to an aqueous fluid and the second internal surface portion has an affinity to an oleic fluid. Because the carrier fluid and the target fluid are immiscible, the first internal surface portion and the second internal surface portion have different fluid affinities. Further, the first internal surface portion and the second internal surface portion are configured to substantially maintain stable fluid flow of the two immiscible fluids in the fluid channel.

As an example, in the embodiment shown in FIG. 7D, an internal surface 41 is configured to have an affinity to a carrier fluid 44. Internal surface or surfaces 42 are configured to have an affinity to a target fluid 45. When immiscible fluids 44 and 45 flow in fluid channel 21, the fluids form a fluid interface 43 which creates a pseudo-membrane.

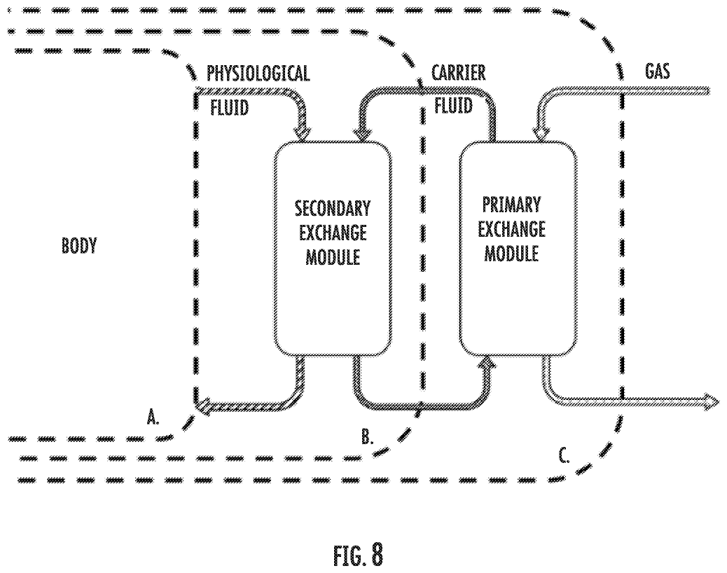

FIG. 8 depicts various possible arrangements of the system 10, relative to the body of a patient. In some embodiments, the two modules/stages of system 10 may be together in a single unit located outside the body of a patient and connected to patient. This arrangement is depicted in Example A of FIG. 8. In this arrangement, the system 10 can be connected to the vasculature by one of many methods, including a double-lumen catheter into the jugular vein or the femoral artery, or a combination of catheters providing input to and output from the device from different veins and/or arteries.

In another example, stages and modules of system 10 may also be together in a single unit and fully implanted in the body of the patient. This arrangement is depicted in Example C of FIG. 8. When the system is fully implanted, it may be implanted in one of a number of locations, including in the chest or in the abdomen. When implanted in the chest, the most likely but not only location is in the right half, replacing the right lung. In this case, the connections to the body's vasculature will be most likely made directly to the pulmonary artery (input to device) and pulmonary vein (output from device) and the air connections can be made through the skin or to the trachea. When implanting in the chest, the system is taking over the space of an organ that is no longer needed, the system has direct access to higher pressure blood, and the connection to air is closer to the natural supply. When implanted in the abdomen, the connection to the body's vasculature is most likely made to the aorta (input to device) and the vena cava (output from device), and the air connections can be made through the skin and/or umbilicus. When implanting in the abdomen, a less traumatic surgery is required as compared with implantation in the chest, and provides for easier removal of the device if that becomes necessary.

Example B of FIG. 8 depicts an arrangement where the exchange modules are separate. In this arrangement, secondary exchange module 20 is positioned within the body and the primary exchange module 12 is positioned outside of the body. In this arrangement, the secondary exchange module 20 may be positioned in the body, such as in the abdomen or in the chest, in a similar fashion as described above with reference to Example C. To connect the primary exchange module outside of the body to the secondary exchange module implanted in the body, tubing or other means are incorporated for allowing flow of the carrier fluid between the two stages. For example, when implanted in the chest, the connections from the secondary exchange module to the body's vasculature is to the pulmonary artery (input to module) and pulmonary vein (output from module), and the tubing to the primary exchange module passes through the center of the patient's chest or through the abdominal wall by tunneling the tubing. In another example, when implanted in the abdomen, the connections from the secondary exchange module to the body's vasculature is to the aorta (input to module) and vena cava (output from module), and the tubing to the primary exchange module passes through the patient's abdominal wall.

Positioning only the secondary exchange module within the patient's body results in a less traumatic and safer implantation as compared with positioning the entire system within the body. As compared with positioning the entire system outside of the body, this arrangement is safer because it decreases the movement and jostling of the blood which decreases the likelihood of bleed out and minimizes the risk of disconnecting the blood path. This also reduces the risk of infection since the connection to the vascular system is completely internal. Finally, in this arrangement, the blood does not cool as it travels to and from the system.

### Single-Stage Exchange System

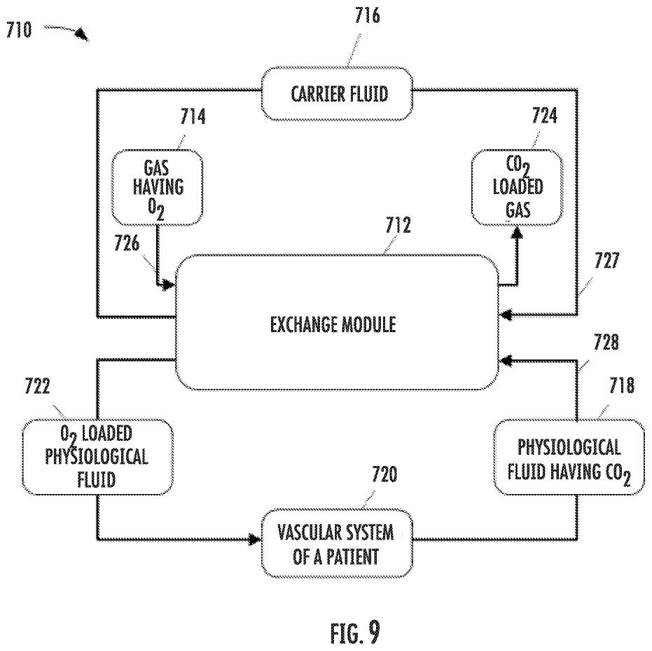

Referring now to FIG. 9, a block diagram of a single-stage system 710 for exchanging small molecules with a fluid, particularly for oxygenating and removing carbon dioxide from a physiological fluid, is shown. System 710 differs from system 10 of FIGS. 1-7D in that system 710 includes only a single exchange module 712 where the oxygen and carbon dioxide exchanges occur.

As shown in FIG. 9, system 710 includes exchange module 712 having an exchange chamber configured to receive a first fluid, a carrier fluid, and a target fluid (i.e., a fluid that is the target for the small molecule exchange). For example, gas 714 having oxygen therein, carrier fluid 716, and physiological fluid 718 having carbon dioxide therein. The gas 714 is received from a small-molecule conduit, or gas conduit 726, the carrier fluid 716 is received from a carrier fluid conduit 727, and the physiological fluid 718 is received from a target fluid conduit, or physiological fluid conduit 728. In one example, the gas 714 having oxygen therein may include ambient air, an oxygen gas, or any gas having oxygen therein. Carrier fluid 716 is preferably immiscible with respect to physiological fluid 718 and has a high capacity for carrying the gas of interest, such as oxygen. In some embodiments, the carrier fluid 716 is made of a perfluorocarbon, such as a perfluorodecalin (C₁₀F₁₈), or similar type compound known to those skilled in the art, that prevents carrier fluid 716 from mixing with physiological fluid 718 having carbon dioxide therein. The carrier fluid 716 must also be able to carry carbon dioxide. Physiological fluid 718 may include blood, serum, or any similar type physiological fluid having carbon dioxide therein. In one example, physiological fluid 718 having carbon dioxide therein may be received from vascular system of a patient 720.

Exchange module 712 transfers oxygen from gas 714 having oxygen therein to carrier fluid 716, and transfers the oxygen from oxygen loaded carrier fluid 716 to physiological fluid 718 to produce oxygen loaded physiological fluid 722. Simultaneously, carbon dioxide from physiological fluid 718 is transferred to the carrier fluid 716 and the carbon dioxide in carrier fluid 716 is transferred to gas 714 to create carbon dioxide loaded gas 724. The stream of carrier fluid 716, as a result of the continuous gas transfer, is an oxygen-enriched stream. For example, the stream may be 100% oxygen or may be a 40% oxygen/60% nitrogen stream. Carbon dioxide loaded gas is preferably expelled from exchange module 712. Oxygen loaded physiological fluid 722, which now has carbon dioxide removed, is transferred to vascular system of a patient 720.

As a result, system 710, through exchange module 712, receives physiological fluid 718 having carbon dioxide therein, effectively removes carbon dioxide therefrom and loads physiological fluid 718 with oxygen. Oxygen loaded physiological fluid 722 may be then transferred to vascular system of patient 720. Thus, system 710 can be used to effectively assist or replace the function of diseased or damaged lung(s). In one embodiment, system 710 may be used as an artificial lung.

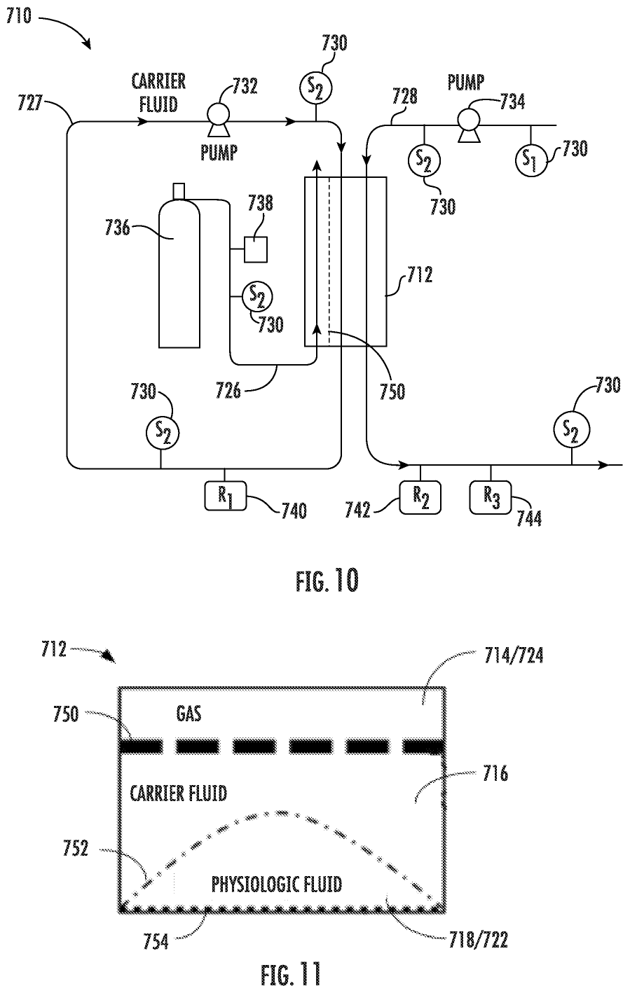

A schematic diagram of an embodiment of system 710 is shown in FIG. 10, showing additional elements of the system 710 which enable the flow of fluid therethrough. In the embodiment shown, system 710 includes a plurality of sensors 730 which are used to sense the flow of fluid through the system to confirm proper flow rates and/or analyze flow through the system. In this embodiment, system 710 also includes carrier fluid pump 732 and physiological fluid pump 734 for moving the fluids through the system. The gas 714 having oxygen is provided to the system 710 by a gas source 736 which may be compressed and use, for example, a blower or a vacuum to provide gas 714 to the system 710. The flow of the gas 714 from the gas source 736 is controlled by regulator 738. Finally, the system 710 shown in this embodiment includes a plurality of reservoirs, such as physiological fluid catcher 740 in the carrier fluid 716 stream, air bubble reservoir 742, and carrier fluid catcher 744 in the physiological fluid 722 stream.

FIG. 10 also depicts a stacked flow of fluid within the exchange chamber of exchange module 712. As shown, the gas 714/724, carrier fluid 716, and physiological fluid 718/722 flow parallel to one another within module 712. In the embodiment shown, the gas 714 flows in the opposite direction of the flow of carrier fluid 716 and physiological fluid 718. Opposing flow increases the efficiency of the system 710, however, flow of gas 714 in the same direction of the carrier fluid 716 and physiological fluid 718 is possible. There is a gas-permeable membrane 750 positioned between the gas 714 and carrier fluid 716 flows.

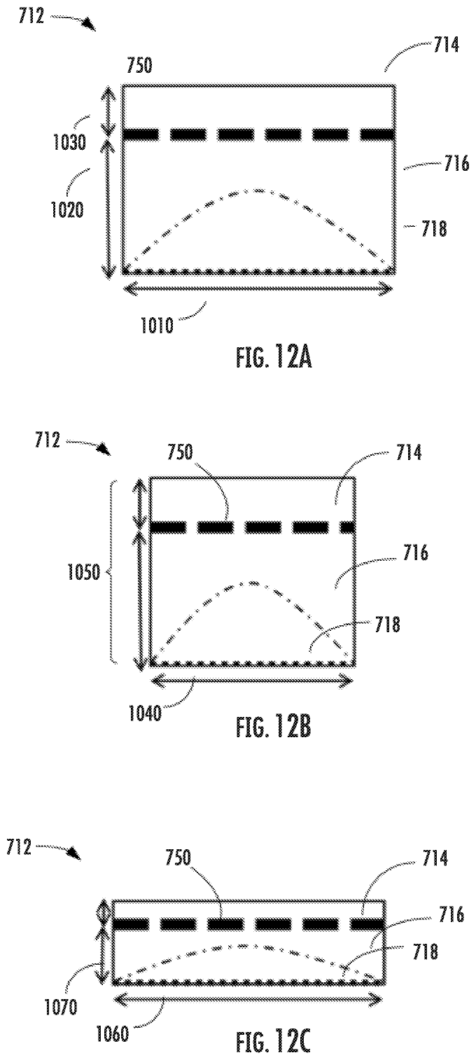

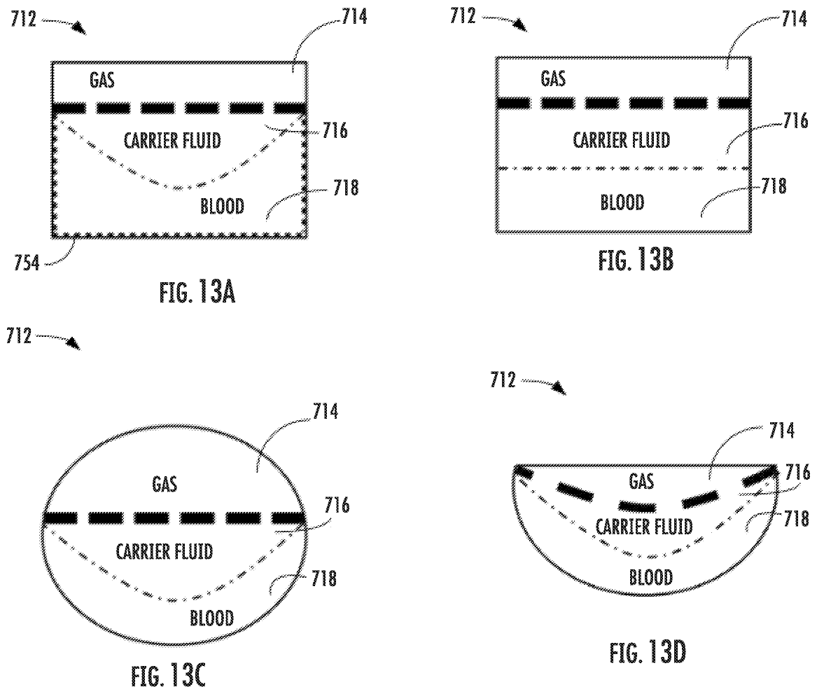

A cross-sectional view of the exchange chamber of exchange module 712 is shown in FIG. 11, showing the stacked flow in greater detail. For clarity, it is noted that the fluids depicted in FIG. 11 (and FIGS. 12A-13D that follow) are flowing in a direction into and out of the paper, and not laterally side-to-side. As shown, a membrane 750 is disposed within the exchange chamber so as to be positioned between the gas 714 and the carrier fluid 716, and permit the transfer of the oxygen and the carbon dioxide between the gas and the carrier fluid. The material of the membrane 750 is compatible with both the gas 714 and the carrier fluid 716 (for example, it should not corrode in the presence of either), and it should be permeable to the gases of interest without allowing the carrier fluid to leak through. This may be achieved by material properties or by properties of the processing of the material. For example, there are some materials that will naturally allow the diffusion of certain gas molecules while other materials, if processed a certain way and at a certain thickness, will have the desired properties. It is also possible that this material does NOT have the same properties from both directions—it may have one set of properties from the carrier fluid side and another set from the gas side. In some embodiments, the membrane 750 may be positioned between the carrier fluid 716 and the physiological fluid 718, instead of or in addition to the membrane positioned between the gas 714 and the carrier fluid 716.

As described above, the carrier fluid 716 and physiological fluid 718 are preferably immiscible, and accordingly, it is shown in FIG. 11 that the two fluids are in contact (as depicted by meeting line 752), but do not mix. Furthermore, similar to the fluid channel 21 described above with respect to FIG. 6-7D, exchange module 712 may include surfaces coated with, or made of, a material configured to stabilize and further separate the parallel flow of carrier fluid 716 and physiological fluid 718. For example, one of the surfaces that contacts the carrier fluid 716 may be coated, or made of, a material having hydrophobic properties which attracts carrier fluid 716 and repels physiological fluid 718. Other surfaces that are to contact the physiological fluid 718 may be coated with, or made of, a material having hydrophilic properties which attract physiological fluid 718 and repel the carrier fluid 716. For example, FIG. 11 shows one embodiment with the location of a hydrophilic surface 754 along one side wall of the exchange module 712. Such a design stabilizes and further separates the parallel flow of carrier fluid 716 and physiological fluid 718. Furthermore, this design results in the flow of physiological fluid having a dome-shaped profile, as shown by meeting line 752.

The exchange chamber of exchange module 712 is sized and dimensioned for the efficient flow of the fluids through the module. According to various embodiments, using the dimension indicators in FIG. 12A for reference, the width 1010 of the exchange chamber is between 0.5 mm and 10 mm, the height 1020 of the fluid portion of the chamber is between 0.1 mm and 1.0 mm, and the height 1030 of the gas portion of the chamber is between 0.1 mm and 0.5 mm. In an exemplary embodiment, the width is 1.5 mm, the height of the fluid portion is 0.75 mm, and the height of the gas portion is 0.25 mm. According to various embodiments, the flow rate of the gas is between 0.1 mL/min and 10 mL/min, the flow rate of the carrier fluid is between 0.1 mL/min and 4 mL/min, and the flow rate of the physiological fluid is between 0.1 mL/min and 4 mL/min. In an exemplary embodiment, the flow rate of the gas is 1.0 mL/min, the flow rate of the carrier fluid is 0.75 mL/min, and the flow rate of the physiological fluid is 0.75 mL/min

FIGS. 12A-12C depict various embodiments employing different dimensions which are all suitable for the desired fluid flow. In the example shown in FIG. 12A, the liquid portion of the chamber has a width 1010 of approximately 1.5 mm and a height 1020 of approximately 0.75 mm. The gas portion of the chamber is also 1.5 mm wide and is approximately 0.1 mm tall (1030). The dimension of the gas portion of the chamber does not need to be very large for the gas to flow through it. In other examples, the liquid channel is 0.6 mm wide and 1.0 mm tall, however, gas exchange is more efficient where the channel is greater in its width dimension 1010 than in height 1020. In particular, in preferred embodiments, the width 1010 to height 1020 ratio of the fluid portion of the chamber is 2:1. In general, a chamber that is more flat will provide better gas exchange, but maintaining separation of the two liquids will be more difficult, as, for example, the physiologic fluid is more likely to touch the gas permeable membrane. The example chamber shown in FIG. 12B is nearly square, where the width 1040 and height 1050 are substantially the same. The example chamber in FIG. 12C shows an embodiment where the width 1060 to height 1070 ratio for the liquid portion of the chamber is 5:1. Such dimensions are approaching the practical limitation on flow stability, which is difficult to maintain at this ratio.

Despite the liquid channel being of small size, the system is tolerant of pulsatility. The system can be run using peristaltic pumps for both the physiologic fluid and the carrier fluid. It is not necessary that the two utilize the same peristaltic pump, or the same type of pump, each can be presented with different pulsatility profiles. The system has been tested with both fluids on pulsatile pump heads pulsing up to 50% of average pulsations, and the two fluids out of synch. The system is functional without pulsatility in the flow, using centrifugal or impeller pumps for long term flow or syringe pumps for short term flow.

Inside the chamber, where the physiological fluid and carrier fluid are in fluidic contact, they are at essentially the same pressure. The gas pressure in the chamber depends on the properties of the membrane 750 and to a lesser extent on the pressure of the carrier fluid. Some membranes 750 will allow discrete bubbles of gas to form if the gas pressure is too high relative to the liquid, or liquid to leak through if its pressure is too high relative to the gas, so the pressure needs to be adjusted accordingly. Accordingly, the most likely material for the membrane 750 is a silicone, which is less sensitive to these incursions and leaves the gas pressure largely independent of the pressure of the carrier fluid.

FIGS. 13A-13D show various alternative embodiments of the exchange chamber of the exchange module 712 and different flow profiles. In FIG. 13A, the hydrophilic surface extends along three surfaces of the chamber. In this way, carrier fluid 716 contacts only the membrane 750 and is not in contact with any other surface of the chamber. FIG. 13B depicts a linear layering of the carrier fluid 716 and physiological fluid 718, which is likely to occur in a case where the two liquids are miscible, for example if the carrier fluid is a saline, for example, rather than a perfluorocarbon. The system 710 may also alternatively use an exchange chamber having a non-rectangular configuration. For example, FIGS. 13C-13D depict non-rectangular exchange chambers and the resulting flow profile achieved with such configurations.

What is claimed is:

1. An apparatus for exchanging small molecules with a fluid, comprising:
a small-molecule conduit for providing a first fluid having a first type of small molecule;
a target fluid conduit for providing a target fluid having a second type of small molecule therein;
a carrier fluid conduit for providing a carrier fluid that is configured to at least one of: (i) receive at least some of the first type of small molecule from the first fluid and transfer at least some of the first type of small molecule to the target fluid and (ii) receive at least some of the second type of small molecule from the target fluid and transfer at least some of the second type of small molecule to the first fluid;
an exchange module having an exchange chamber in fluid communication with the small-molecule conduit, the target fluid conduit and the carrier fluid conduit to receive the first fluid, the carrier fluid, and the target fluid with the exchange chamber, wherein the exchange chamber is configured (i) to position the first fluid relative to the carrier fluid to permit the transfer of at least one of the first type of small molecule and the second type of small molecule between the first fluid and the carrier fluid and (ii) to position the carrier fluid relative to the target fluid to permit the transfer of at least one of the first type of small molecule and the second type of small molecule between the target fluid and the carrier fluid.

2. The apparatus of claim 1, further comprising a membrane disposed between the small-molecule conduit and the carrier fluid conduit.

3. The apparatus of claim 1, further comprising a membrane disposed between the carrier fluid conduit and the target fluid conduit.

4. The apparatus of claim 1, wherein the first type of small molecule is oxygen and the second type of small molecule is carbon dioxide.

5. The apparatus of claim 1, wherein the first fluid is a gas.

6. The apparatus of claim 1, wherein the carrier fluid is a liquid.

7. The apparatus of claim 1, wherein the target fluid is a physiological fluid.

8. The apparatus of claim 7, wherein the physiological fluid is a liquid.

9. The apparatus of claim 1, wherein the exchange module is configured for the first fluid, the carrier fluid, and the target fluid to flow in parallel paths.

10. The apparatus of claim 1, wherein the small-molecule conduit is in communication with a first side of the exchange module and the carrier fluid conduit and target fluid conduit are in communication with a second, opposing side of the exchange module, such that the flow of the first fluid is in an opposite direction of the flow of the carrier fluid and the target fluid.

11. The apparatus of claim 1, wherein at least one inner surface of the exchange chamber comprises a material having hydrophobic or hydrophilic properties.

12. The apparatus of claim 11, wherein at least one inner surface of the exchange chamber comprises a hydrophilic material which attracts the target fluid and repels the carrier fluid.

13. The apparatus of claim 12, wherein the hydrophilic material comprises polyhydroxyethylmethacrylate.

14. The apparatus of claim 12, wherein more than one inner surfaces of the exchange chamber comprise the hydrophilic material.

15. The apparatus of claim 1, wherein the membrane is gas-permeable.

16. The apparatus of claim 15, wherein the gas-permeable membrane comprises silicone.

17. The apparatus of claim 1, wherein the width to height ratio of the exchange chamber is 2:1.

18. A method of exchanging small molecules with a fluid, comprising:
flowing, through an exchange chamber of an exchange module on a first side of a membrane, a first fluid comprising a first type of small molecules;
flowing, through the exchange chamber of the exchange module on a second side of the membrane, a target fluid having a second type of small molecules therein;
flowing, through the exchange chamber of the exchange module on the second side of the membrane and between the target fluid and the membrane, a carrier fluid that at least one of: (i) receives through the membrane at least some of the first type of small molecules from the first fluid and transfers at least some of the first type of small molecules to the target fluid and (ii) receives at least some of the second type of small molecules from the target fluid and transfers through the membrane at least some of the second type of small molecules to the first fluid,
wherein the first fluid, the target fluid, and the carrier fluid are flowed simultaneously through the exchange chamber of the exchange module.

19. The method of claim 18, wherein the first type of small molecules is oxygen and second type of small molecules is carbon dioxide.

20. The method of claim 18, wherein the first fluid is a gas.

21. The method of claim 18, wherein the carrier fluid is a liquid.

22. The method of claim 18, wherein the carrier fluid comprises perfluorocarbon.

23. The method of claim 18, wherein the target fluid and the carrier fluid are immiscible.

24. The method of claim 18, wherein the target fluid is a physiological fluid.

25. The method of claim 22, wherein the physiological fluid is a liquid.

26. The method of claim 22, wherein the physiological fluid is blood.

27. The method of claim 22, further comprising:
receiving the physiological fluid from a vascular system of a patient for flowing through the exchange chamber of the exchange module; and
after the transfer of oxygen to the physiological fluid, transferring the physiological fluid to a vascular system of a patient.

28. The method of claim 18, wherein the first fluid, the target fluid, and the carrier fluid flow in parallel through the exchange chamber of the exchange module.

29. The method of claim 18, wherein the first fluid flows in a first direction and the target fluid and the carrier fluid flow in a second direction, opposite the first direction.

30. A method of exchanging small molecules with a fluid, comprising:
providing a primary exchange module configured to:
receive a first fluid having a first type of small molecule therein;
receive a carrier fluid having second type of small molecule therein, and
transfer at least one of: (i) the first type of small molecule from the first fluid to the carrier fluid and (ii) the second type of small molecule from the carrier fluid to the first fluid to create at least one of a carrier fluid loaded with the first type of small molecule and a first fluid loaded with the second type of small molecule;
providing a secondary exchange module configured to:
receive the carrier fluid loaded with the first type of small molecule;
receive a target fluid having the second type of small molecule therein; and
transfer at least one of: (i) the first type of small molecule from the carrier fluid loaded with the first type of small molecule to the target fluid and (ii) the second type of small molecule from the target fluid to the carrier fluid to create at least one of a target fluid loaded with the first type of small molecule and a carrier fluid loaded with the second type of small molecule;
implanting the secondary exchange module within a body of a patient; and
positioning the primary exchange module external to the body of the patient.

31. The method of claim 30, wherein the first type of small molecule is oxygen and second type of small molecule is carbon dioxide.

32. The method of claim 30, wherein the first fluid is a gas.

33. The method of claim 30, wherein the carrier fluid is a liquid.

34. The method of claim 30, wherein the target fluid is a physiological fluid.

35. The method of claim 34, wherein the physiological fluid is a liquid.

36. The method of claim 34, further comprising:
coupling the secondary exchange module to a vascular system of the patient; and
coupling the secondary exchange module to the primary exchange module using tubing passing from within the body to an area external to the body.

37. The method of claim 34, wherein implanting the secondary exchange module within the body of the patient comprises:
implanting the secondary exchange module in a chest of the patient;
coupling the secondary exchange module to the vascular system of the patient by coupling an input to the secondary exchange module to the pulmonary artery and coupling an output from the secondary exchange module to the pulmonary vein; and
coupling the secondary exchange module to the primary exchange module using tubing passing through the front of a chest of the patient or an abdominal wall of the patient.

38. The method of claim 34, wherein implanting the secondary exchange module within the body of the patient comprises:
implanting the secondary exchange module in an abdomen of the patient;
coupling the secondary exchange module to the vascular system of the patient by coupling an input to the secondary exchange module to the aorta and coupling an output from the secondary exchange module to the vena cava; and
coupling the secondary exchange module to the primary exchange module using tubing passing through an abdominal wall of the patient.
